(19) United States
(12) Patent Application Publication
(10) Pub. No.: US 2018/0235554 A1
(43) Pub. Date: Aug. 23, 2018

(54) Patient-Specific Restraining Device and Integrated Dosimetry System

(71) Applicant: Eric A Burgett, Pocatello, ID (US)

(72) Inventor: Eric A Burgett, Pocatello, ID (US)

(21) Appl. No.: 15/958,595

(22) Filed: Apr. 20, 2018

Related U.S. Application Data
(60) Provisional application No. 62/459,658, filed on Feb. 16, 2017.

(51) Int. Cl. A61B 6/08; A61N 5/10; A61B 6/03; B33Y 50/02; B33Y 80/00

(52) U.S. Cl. CPC A61B 6/08; A61N 5/1031; A61N 5/1049; B33Y 80/00; A61N 2005/1094; B33Y 50/02; A61B 6/032

(57) ABSTRACT

A patient-specific, restraining device is fabricated directly from the patient's diagnostic images, (e.g. Computed Tomography (CT), Magnetic Resonance Imaging (MRI), and Ultrasound) fabricated by additive manufacturing techniques, also known as 3D printing, using an algorithm that directly translates the medical images into instructions for the 3D printer. The patient-specific restraining device incorporates dosimetry devices to allow for real-time, near real-time or after-the-fact measurement of delivered radiation at the entry and exit points on the body. Using a patient's medical images and dose planning software, patient-specific dose calculations allows for the calculation of the predicted dose for each location of entry and exit of the beam on the restraining device. This restraining device and method may be used to measure irradiation dosages in real time, adjust dosage levels based on such measurements, and then deliver more accurate and precise treatments during advanced treatment techniques.

### BACKGROUND OF THE INVENTION

One of the most common radiation therapy modalities is external beam radiation therapy. In this type of radiation therapy, a beam of radiation is created with an electron accelerator for electrons, a proton accelerator for protons, or a radioactive source for gamma rays. In current external beam radiation therapy techniques, the radiation beam is shaped to maximize the dose to the active cancer volume and minimize the dose to the healthy tissues. The patient is often held in place with a restraining device which aids in the alignment of the patient and immobilizes the treatment area to maintain that alignment during treatment. The present invention relates generally to a system and method for improving the effective delivery of radiation therapy and for improving the quality assurance/quality control (“QA/QC”) of advanced radiation treatment techniques. This is accomplished through the use of a patient-specific restraining device created through additive manufacturing techniques into which high spatial resolution dosimeters are integrated to achieve real-time or near real-time measurement of delivered radiation dose.

Radiation therapy of cancers has evolved significantly over the last few decades. Advances in treatments have strived to minimize the radiation dose delivered to the healthy tissue surrounding the active cancer volume while maximizing the efficacy of the dose delivered to the actual cancer. This has been accomplished through advances in high resolution imaging, e.g., Computed Tomography (“CT”), Magnetic Resonance Imaging (“MRI”), and Ultrasound combined with advanced radiation treatment techniques that: increase the number of treatment fields and alter the directions in which the radiation is externally applied, e.g., intensity-modulated radiation therapy, volumetric modulated arc therapy, and dynamic continuous arc therapy; or that time-gate the application of the radiation, e.g., real-time tumor tracking radiotherapy and respiration gated radiation therapy. With the increase in complexity of radiation treatments, more sophisticated QA/QC techniques are needed to ensure effective treatment while maintaining patient safety. In many instances, existing QA/QC techniques are not adequate for these advanced treatment techniques and new techniques are required. A patient-specific restraining device has been created that uses air-equivalent polymer materials, additive manufacturing techniques (i.e., 3D printing), and the incorporation of real-time or near real-time radiation dosimetry devices that together can improve the efficacy and safety of modern radiation therapy. The air equivalence ensures that the patient-specific restraining device does not shield, alter, or reduce in a statistically significant way the delivered radiation dose to the patient.

The current standard of care involves placing the patient on a treatment surface (e.g., a bed or couch) and transferring a coordinate system onto the patient; typically a series of marks are drawn on the patient's skin and used to align the patient and the restraining system with respect to the radiation beam. All of the currently-available restraint systems were designed only to verify and maintain patient location during treatment. They do not provide any information or feedback to the radiation delivery system or system operators during treatment. The current standard of care also involves fitting some patients with a custom-fit vacuum lock bed and/or a thermoset plastic mesh which is placed over the patient at the beginning of the treatment. This combination aids in the alignment of the patient over the course of treatment.

The treatment dose is computed using a non-patient-specific phantom and a calibrated ion chamber to correlate the output of the accelerator to a dose received by the calibrated ion chamber. The treatment plan will typically be delivered in multiple sessions over a period of 10-30 days. During this time, the patient's body can experience changes such as weight loss, loss of muscle tone, etc. The patient's alignment is verified at the beginning of each treatment session. However, these current methods do not use real-time, patient-specific dosimetry during the treatment so there is no way to assure that the radiation dose is consistently aligned with the cancer volume; or that the amount of radiation intended to be delivered to the cancer volume has in fact been delivered as required.

With the introduction of more sophisticated treatment techniques, such as intensity-modulated radiation therapy (“IMRT”) and dynamic continuous arc therapy, traditional QA/QC tools and techniques are inadequate particularly for patients whose cancer volume changes or moves dynamically during the course of treatment. To compensate, physicians typically treat a larger than necessary volume of healthy tissue surrounding the cancer. This causes non-cancerous, healthy tissue to be irradiated unnecessarily, thereby increasing the chances that a patient will suffer secondary cancers or other damage to healthy tissues. With the increase in complexity of radiation treatments, more sophisticated QA/QC techniques are needed to ensure effective treatment and patient safety while reducing the possibility of secondary cancers and other negative effects. The present invention of a patient-specific restraining device with integrated dosimetry can more effectively accomplish these aims than current methods.

### BRIEF DESCRIPTION OF THE DRAWINGS

These and other features, aspects, and advantages of the present invention will become better understood with regard to the following description, appended claims, and accompanying drawings where:

FIG. 1 is a side view of one embodiment of a non-field perturbing dosimeter.

### DESCRIPTION OF THE PATIENT-SPECIFIC RESTRAINING DEVICE

A patient-specific, restraining device is fabricated directly from the patient's diagnostic images, (e.g. Computed Tomography (CT), Magnetic Resonance Imaging (MRI), and Ultrasound) fabricated by additive manufacturing techniques, also known as 3D printing, using an algorithm that directly translates the medical images into instructions for the 3D printer.

The restraining device consists of a cross-hatched mesh pattern that covers the necessary portion of the patient. The mesh pattern has sufficient polymer to provide rigidity to the device to aid in maintaining proper positioning of the patient and adequate open spaces to allow for patient comfort.

The restraining device is preferably printed using high Shore Hardness, air-equivalent polymers. Air equivalent polymers have low densities, high air entrainment, and low Z numbers to minimize the effect of the device on the radiation beam. Polymers suitable for the restraining device include thermoset polymers, multi-part resins, vinyls, urethanes, and elastomers which are binary, ternary, or multi-part, including a resin base and a hardener and can be polymers of acrylates, ethylenes, esters, and the like, e.g., acrylonitrile butadiene styrene (ABS), polylactic acid (PLA), polyvinyl alcohol (PVA), polymethyl methacrylate (PMMA), high-density polyethylene (HDPE), polyethylene (PE), low-density polyethylene (LDPE), and fluorinated and chlorinated plastics like polyvinyl chloride (PVC), polytetrafluoroethylene (PTFE), polystyrenes, and other suitable materials may be used.

The patient-specific restraining device incorporates dosimetry devices to allow for real-time, near real-time or after-the-fact measurement of delivered radiation at the entry and exit points on the body. Several different types of radiation dosimeter can be used, including: miniature non-field perturbing ion chambers; direct-reading radio-chromic polymer dosimeters; secondary direct-reading dosimeters; and non-direct-reading dosimeters.

Description of a Patient-Specific Restraining Device with Integrated Miniature, Non-Field Perturbing Ion Chambers

In this embodiment, the real-time patient-specific restraining device incorporates miniature, non-field perturbing radiation dosimeters made of air-equivalent polymers that have effectively no influence on the measured radiation field compared to current measurement techniques and can be precisely placed in the specific locations of the restraint that requires measurement. Utilizing the patient medical images (e.g. Computed Tomography scan) and the proposed treatment plan design, the restraining device incorporates matched pairs of ion chambers that are placed on directly opposite sides of the patient which correspond to the inlet and outlet fields on the patient. For example, in a conventional five field treatment plan, the restraining device would incorporate ten dosimeters; one pair for each treatment field. The dosimeter placements are precisely chosen to maximize the resolution of the measurement of the incoming radiation and the exiting radiation. For N treatment fields (conventional IMRT therapy), 2N dosimeters are placed around the patient; one for the entrance and one for the exit of each field aligned in the plane normal to the treatment plane. For continuous arc radiation therapy, a continuous band of ion chambers in the treatment plane is utilized and individual pixel planes are activated as the treatment plane becomes normal with respect to the detectors.

Description of the Non-Field Perturbing Miniature Ion Chambers

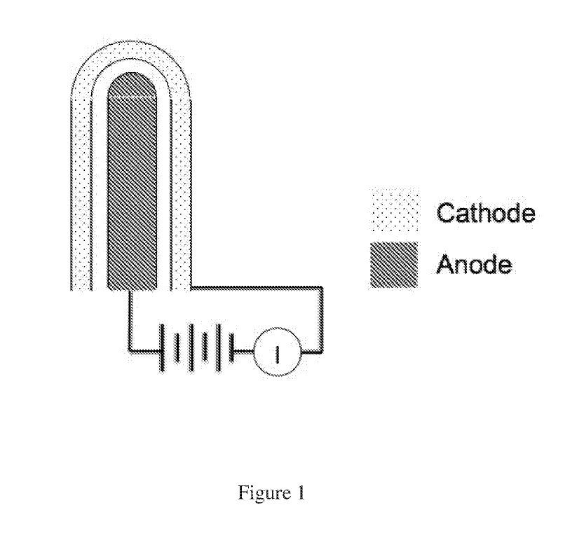

As depicted in FIG. 1, the non-field perturbing dosimeters are created using air-equivalent polymers with low densities, high air entrainment, and low Z number to minimize the perturbation of the photon fields. In one embodiment, a chamber wall [1] is made from this air equivalent material. The materials used to make the chamber wall [1] and central terminal can be constructed of from these air-equivalent materials that are made conducting through the inclusion of carbon materials. The chambers may also include a set of one or more coaxial cables to apply a voltage potential to the dosimeter and to provide an output signal of dosimetry data. The coaxial cables may also be connected to electrometer or precision capacitor which can be multiplexed to read out numerous dosimeters simultaneously. Alternatively, a very thin metallic layer can be deposited onto the dosimeter chamber's interior wall. The anode [3] and cathode [4] of these miniature dosimeters are electrically connected to the instrumentation system and made direct-reading either through interconnect wires or through wire-bonded or printed circuit traces made with conductive polymers deposited on the restraint structure layers themselves during the 3D printing process. Functioning as a small, gas-filled ionization chamber [5] (<0.1 cubic centimeter), radiation-induced ionization events produce ion-electron pairs in the chamber. A small applied voltage between the anode and cathode allows the ion-electron pairs to be collected on the anode and cathode. This collected charge is then read out on a micro-, nano-, or pico-ammeter depending on the amount of charge collected. The charge collected is proportional to the dose delivered and responds linearly with increases in total dose and dose rate.

Description of a Patient-Specific Restraining Device Made from Direct-Reading Radio-Chromic Polymers

Another embodiment of this invention is a patient-specific restraining device fabricated using radio-chromic compounds mixed with a transparent polymer matrix. In this embodiment the entire restraining device serves as radiation dosimeter. Polymers suitable for the matrix must be transparent and be air-equivalent with low densities, high air entrainment, and low Z number to minimize the perturbation of the photon fields. Polymers such as such as polymethyl methacrylate (PMMA), polycarbonate, or transparent vinyl including multi-part resins and/or elastomers which are binary, ternary, or multi-part including a resin base and a hardener can be used. Radio-chromic chemicals such as diarylethenes, azobenzenes, and phenoxynaphthacene quinone, as well as metal halides including but not limited to zinc halides and silver halides are added to the polymer matrix formulation during fabrication. The radio-chromic compounds change color proportionally to the absorbed ionizing radiation dose to the air-equivalent materials. In this embodiment the entire restraining device made from the radio-chromic polymer would serve as the radiation dosimeter. In this embodiment, the restraining device (as the dosimeter) would have to be read after the dose is received and would not provide real-time dose information; rather near-real time dose information. These three-dimensional patient-specific restraining devices are printed from the above mentioned polymer materials in a 3D printer.

After fabrication is completed, the restraining device is transparent, or nearly so. Following irradiation, the radio-chromic materials darken and change color proportionally to the absorbed radiation dose. The restraining device is then digitally imaged in a medium such as in air or in another fluid with an index of refraction matching the phantom to record the colors within the phantom. This digitized image data can then be overlaid on an image of the patient's anatomy to create a composite image that maps dose received during the treatment to specific locations in the patient's treatment area. This technique provides 100% imaging coverage of the cancer area and surrounding tissues enhancing understanding of delivered dose. Imaging and quantification are accomplished through optical systems such as digital optical scanning and optical filtering or through directed laser scanning of the peak wavelengths of absorption. Three dimensional scanning is completed and reconstructed using standard commercially-available computer software. The restraining device will transition back to transparency after a decay period allowing for reuse.

Secondary Direct Reading Dosimetry Options

A third embodiment has the matched pairs of dosimeters made from a solid-state material comprising a semiconductor diode dosimeter operating in pulse or continuous current modes, or a solid-state crystalline scintillator which can be fiber optically coupled to a readout device. In this embodiment, the patient-specific restraining device would be fabricated as described above using 3D printing and air-equivalent polymers. The placement of the dosimeters would occur in matched pairs as described for the miniature non-field perturbing ion chambers above.

Non-Direct Reading Dosimetry Options

A fourth embodiment would use non-direct reading dosimetry options such as solid-state crystalline, amorphous, or powdered material which stores absorbed ionizing radiation in its crystalline lattice and can be read out after irradiation, such as an Optically Stimulated Luminescence (“OSL”) dosimeter or a Thermos-Luminescent Dosimeter (“TLD”). In this embodiment, the patient-specific restraining device is fabricated as described above using 3D printing and air-equivalent polymers. In this embodiment, the placement of the dosimeters would occur in matched pairs as described for the miniature non-field perturbing ion chambers above. In this embodiment, the dosimetry would provide only after-the-fact confirmation of delivered dose; not real-time or near-real time information.

Improved Real Time Data Acquisition

Using the matched pairs of real-time dosimeters at the radiation beam entrance into and exit from the patient, real-time corrections can be calculated and corrections can be made to the dose delivery device. Using a patient's medical images and dose planning software, patient-specific dose calculations can be made that allow the predicted dose for each location of entry and exit of the beam on the restraining device to be calculated, accounting for shadowing effects of organs, bones, and tissue. With the high resolution of this system, and the nature of IMRT based radiation, these patterns can be pre-calculated for each specific treatment segment or position. Then, as dose measurements are made by the dosimeters, these data can be automatically compared to the expected output in real-time. Deviations from expected results can be used to control the radiation source so that treatment can be automatically stopped, an alert or alarm sounded, and/or the patient repositioned, as appropriate. The total dose for each fraction of the treatment can be quantified in real-time for every dose, every field, and every time segment, pulse by pulse of the accelerator. This prevents overdosing conditions due to hardware or software errors that can occur.

Improved Time Gating of Time-Gated, Intensity-Modulated Radiation Therapy

With the advancement of the capabilities of time-gated, intensity-modulated radiation therapy, an improved QA/QC system is needed to verify the time-dependent delivery on a patient-specific basis. Snug-fitting soft polymers are used to print the support structure of the restraint devices. This produces a softer, more pliable patient restraint device allowing for additional flexibility during patient breathing and allowing limited motion.

The soft, pliable polymers are created by modifying the material formulation of the air-equivalent materials. A secondary means of measurement of motion is utilized such as through the attachment of the restraining device to an integrated readout base with the incorporation of mechanical micro-strain gauges or inclusion of optical strain gauges (see U.S. patent application Ser. No. 14/808,896, which is incorporated herein by reference). As opposed to obtaining air-equivalent materials from high-rigidity polymers with high Shore Hardness, low Shore Hardness material formulations, e.g. ethylene propylene diene monomer (“EDPM”), silicone rubbers, and others, are used to create a pliable and deformable restraining device. These soft polymers can mimic a patient's body type and natural respiratory behavior while minimizing the energy-dependent effective Z numbers. This restraining device can include integrated matched pairs of radiation dosimeters as described above or can be alloyed with radio-chromic polymer compounds that change colors during irradiation as described above. This provides radiation dose response with patient-specific tissue morphologies as a function of time.

Polymer formulations for the time-dependent dosimeter restraining device include multi-part resins and/or elastomers which are binary, ternary, or multi-part including a resin base and a hardener. Restraining device polymer densities are controlled through the addition of air, water, solvents, ethylene, and/or other materials to achieve minimum air-like densities and energy-dependent effective Z numbers. In this embodiment of the invention, the elastic properties of the polymer material(s) are tuned to more closely represent that of the patient's tissues and body shapes. The elastic properties are controlled through the precise addition of chemicals used in the formulation as well as a controlled degree of cross-linking and cross-linking techniques including e-beam, ion beam, photon (X-ray), and Ultraviolet (“UV”) curing/cross linking techniques which can be applied during the fabrication process or prior to printing during the extrusion process.

Respiratory function can be quantified in real time by monitoring the strain gauges which indicate the chest cavity inflate/deflate following the lung volume change cycles with air due to breathing. Cardiac function can be quantified through attaching an external ultrasound mechanism to determine the motions of the heart. In these ways, time-dependent physical and deformable characteristics and motions of the body can be accounted for and used to deliver more accurate treatments while simultaneously measuring the delivered doses during advanced treatment techniques.

1. A method for improving the effective delivery of radiation therapy, said method comprising the steps of:
obtaining a high resolution image of a portion of a patient's body that is to be treated with radiation therapy;
translating said high resolution images into instruction for a three dimensional printer;
using said three dimensional printer to fabricate a patient specific restraining device that incorporates dosimetry devices to allow for measurement of delivered radiation at the entry and exit points on the body;
positioning said restraining device on said patient, so that said restraining device covers said portion of said patient's body that is to be treated with radiation therapy;
applying a dose of radiation therapy to said portion of said patient's body, so that said radiation passes through said restraining device;
using said dosimeters to measure radiation levels at an entry point to said patient's body and at an exit point from said patient's body;
comparing actual, measured dosages of radiation with predicted dosages of radiation; and
adjusting ongoing and future radiation dosages based on said comparison between measured dosages and predicted dosages.

2. The method for improving the effective delivery of radiation therapy set forth in claim 1, wherein said restraining device is made from an air equivalent polymer, and is formed into a cross-hatched mesh.

3. The method for improving the effective delivery of radiation therapy set forth in claim 1, wherein said restraining device is formed from a polymer selected from the group consisting of thermoset polymers, multi-part resins, vinyls, urethanes, and elastomers which are binary, ternary, or multi-part, including a resin base and a hardener and can be polymers of acrylates, ethylenes, esters, acrylonitrile butadiene styrene (ABS), polylactic acid (PLA), polyvinyl alcohol (PVA), polymethyl methacrylate (PMMA), high-density polyethylene (HDPE), polyethylene (PE), low-density polyethylene (LDPE), and fluorinated and chlorinated plastics, polyvinyl chloride (PVC), polytetrafluoroethylene (PTFE), and polystyrene, or some combination thereof.

4. The method for improving the effective delivery of radiation therapy set forth in claim 1, wherein said restraining device is made using polymers including radio-chromic compounds that change color based on exposure to radiation, wherein said polymers are selected from the group consisting of polymethyl methacrylate (PMMA), polycarbonate, or transparent vinyl including multi-part resins and/or elastomers which are binary, ternary, or combinations thereof; and
wherein said radio-chromic compounds are selected from the group consisting of diarylethenes, azobenzenes, and phenoxynaphthacene quinone, metal halides, zinc halides and silver halides.

5. The method for improving the effective delivery of radiation therapy set forth in claim 1, further including the step of incorporating matched pairs of dosimeters into said restraining device, wherein said dosimeters are made from a solid-state material comprising a semiconductor diode dosimeter operating in pulse or continuous current modes.

6. The method for improving the effective delivery of radiation therapy set forth in claim 1, further including the step of incorporating matched pairs of dosimeters into said restraining device, wherein said dosimeters are made from a solid-state crystalline scintillator which can be fiber optically coupled to a readout device.

7. A patient specific restraining device for use with advanced radiation therapy treatments, said restraining device comprising:
a polymeric mesh material;
a plurality of dosimetry devices integrated within said polymeric mesh material for measuring dosage levels of delivered radiation at the entry and exit points on a patient's body

8. The patient specific restraining device set forth in claim 7, wherein said restraining device is made from air equivalent polymers, and is formed into a cross-hatched mesh.

9. The patient specific restraining device set forth in claim 7, wherein said restraining device is formed from a polymer selected from the group consisting of thermoset polymers, multi-part resins, vinyls, urethanes, and elastomers which are binary, ternary, or multi-part, including a resin base and a hardener and can be polymers of acrylates, ethylenes, esters, acrylonitrile butadiene styrene (ABS), polylactic acid (PLA), polyvinyl alcohol (PVA), polymethyl methacrylate (PMMA), high-density polyethylene (HDPE), polyethylene (PE), low-density polyethylene (LDPE), and fluorinated and chlorinated plastics, polyvinyl chloride (PVC), polytetrafluoroethylene (PTFE), and polystyrene, or some combination thereof.

10. The patient specific restraining device set forth in claim 7, wherein said dosimetry devices are made from radio-chromic compounds that change color based on exposure to radiation, wherein said polymers are selected from the group consisting of polymethyl methacrylate (PMMA), polycarbonate, or transparent vinyl including multi-part resins and/or elastomers which are binary, ternary, or combinations thereof; and
wherein said radio-chromic compounds are selected from the group consisting of diarylethenes, azobenzenes, and phenoxynaphthacene quinone, metal halides, zinc halides and silver halides.

11. The patient specific restraining device set forth in claim 7, wherein matched pairs of dosimeters are integrated into said restraining device, and wherein said dosimeters are made from a solid-state material comprising a semiconductor diode dosimeter operating in pulse or continuous current modes.

12. The patient specific restraining device set forth in claim 7, wherein matched pairs of dosimeters are integrated into said restraining device, and wherein said dosimeters are made from a solid-state crystalline scintillator having means for fiber optically coupling said dosimeters to a readout device.
